(19) United States
(12) Patent Application Publication
(10) Pub. No.: US 2006/0217320 A1
(43) Pub. Date: Sep. 28, 2006

(54) Soft gel formulations for saquinavir

(75) Inventors: Ogari Pacheco, Itapira (BR); Elisa Russo, Itapira (BR); Valter Russo, Itapira (BR)

Correspondence Address: BIRCH STEWART KOLASCH & BIRCH, PO BOX 747, FALLS CHURCH, VA, 22040-0747, US

(73) Assignee: Cristalia Productos Quimicos Farmaceuticos LTDA, Itapira (BR)

(21) Appl. No.: 10/565,220

(22) PCT Filed: Jul. 19, 2004
(86) PCT No.: PCT/BR04/00118

§ 371 Date: May 22, 2006

(30) Foreign Application Priority Data
Jul. 23, 2003 (BR) PI0302424-5

(51) Int. Cl. A61K 38/05; A61K 31/4709

(52) U.S. Cl. 514/18; 514/310

(57) ABSTRACT

The present invention describes a pharmaceutical composition with increased bioavailability, which is suitable for preparing microcapsules for the therapeutic administration of a protease inhibitor. It is also described a process for preparing a pharmaceutical composition with increased bioavailability, suitable to prepare concentrate pharmaceutical compositions comprising retroviral protease inhibitors.

The present invention describes a pharmaceutical composition with increased bioavailability, which is suitable for preparing microcapsules for the therapeutic administration of a protease inhibitor. It is also described a process for preparing a pharmaceutical composition with increased bioavailability, suitable to prepare concentrate pharmaceutical compositions comprising retroviral protease inhibitors.

The administration of drugs using soft gelatin capsules is a usual practice that is becoming more popular in the last years.

Typically soft gelatin capsules are comprised by a liquid containing the active pharmaceutical ingredient surrounded by an elastic gelatin pellicle. Due its elastic behavior that allows an easier ingestion, its acceptance among patients is higher than the conventional tablets or hard gelatin capsules.

Another important property derived from this kind of formulation is to allow the administration of a medicine wherein the pharmaceutical active ingredient is completely dissolved in a solution. Once ingested, the capsule breaks inside the gastrointestinal tract liberating the medicine in an homogeneous way, and as a liquid, it is not necessary first to be dissolved by the body in order to be absorbed later.

It is known that several active pharmaceutical ingredients show different absorption behavior inside the biological environment, many times beeing dependent from their actual crystalline form.

Polymorphism is a physical-chemical characteristic that is very important in bioabsorption studies, since different crystalline forms of an active pharmaceutical ingredient (active ingredient) usually proportionate solubility differences, and so, different absorption rates by the organism. These absorption differences usually proportionate different plasmatic levels in the blood, conferring an erratic pattern in the minimum circulating level of the drug in the organism. As a consequence of this erratic pattern, those individuals treated are susceptible to subdosages or overdosages of these drugs.

Even subdosages as overdosages are negative therapeutic aspects, as they may lead to a complication in the clinical picture of the patient. In the case of a subdosage, the patient do not receive the minimum necessary dose of the drug for the stabilization or the cure of its disease, while in the case of an overdosage the patient absorbs a higher amount of the drug than the necessary, which can lead to a severe intoxication picture.

There are drugs that present an excellent effect “in vitro” but due their low solubility they are not adequately absorbed “in vivo” beeing considered as therapeutically ineffective.

Soft gelatin capsules allow the administration of several active pharmaceutical ingredients having the above-described characteristics as a pre-concentrate microemulsion, or even in the form of a solution, pharmaceutical presentations that make their absorption easier in the biological environment. Issues like low solubility and different crystalline aspects may be easily controlled by using soft gelatin capsules allowing an adequate, reliable and predictable performance of the drug.

Besides the fact of the active ingredient is administered as a liquid medium, the contained excipients may help in other absorption characteristics, for example, promoting a medium wherein the drug can be absorbed easier because a pH control or because the presence of substances that can assist or promote the transport through specific membranes or cells of a specific part of the organism. These substances, called absorption promoters, can be introduced easily in these pre-concentrate microemulsions making the drug absorption easier by the organism and making the drug more effective.

Especially in the treatment of infections caused by virus or bacteria, a complicating factor is the development of resistant roots a reasonable occurrence when the active drug does not reach the infected target issue in a minimum amount necessary to the eradication or complete inactivation of the infectious agent. Resistance is without any doubt one of the biggest problems that may overcome during the treatment of diseases caused by infectious organisms and the efforts to avoid it are frequent.

There are several issues that a drug must comply in order to be considered adequate as a therapeutic agent. Among them, a drug must present therapeutic efficacy and for that it has to possess adequate characteristics of bioabsorption and bioavailability. Besides that, the drug must present an administration route less invasive as possible in order to avoid the patient to be submitted to painful and uncomfortable procedures during its administration. Another extremely important factor is that it has to stimulate or to motivate the patient adherence or adhesion to the therapy.

Adherence or adhesion to a treatment may be defined as the act, action or quality of being consistent with the administration of the prescribed medicaments. This issue is so more important as longer the time of duration of the therapy prescribed to a patient.

Nowadays, AIDS (Acquired Immunodeficiency Syndrome) treatment uses medicines that must be administered every day, many of them in high dosages and, which treatment can not be interrupted even a day for the rest of the infected individuals life. In the case of AIDS, antiretroviral therapy success depends mainly on the adherence of the patient to the therapy itself, this therapy consisting in the ingestion of significant amounts of medicines several times a day.

Accordingly with the final report named “Avaliacão da Aderência ao Tratamento por Anti-retrovirais em Usuários de Ambulatórios do Sistema Publico de Assistência a AIDS no Estado de São Paulo” (a report evaluating the adherence of patients to antiretrovital therapy in the State of São Paulo—Brazil), non-adherence to new AIDS medicines (antiretrovirals mainly and protease inhibitors in particular) is considered one of the most threatening dangers to the effectiveness of the treatment, in an individual level, and for the dissemination of virus-resistance, in a collective level. That is because new AIDS administration therapies seems to request from the “adherent” a complex integration between knowledge, ability and acceptance, besides other important issues related to the environment and to the health care. The report discloses the matter of non-adherence being a universal phenomenon in a certain rate, occuring either in rich and poor countries, even in cases of life threatening diseases.

Adherence to antiretrovirals is a motive of apprehension among health professionals, once researches about it demonstrate that it is very low even in rich countries, where it reaches only 70% of the patients under treatment (Walsh J., Dalton M., Gill J., Wilkinson D. Burgess A. P., Gazzard B. G.—Adherence to protease inhibitor based highly effective anti-retroviral therapy (HAART) in 12^{th} World Aids Conference, Geneva 1998. Abstracts; Hecht F. M., Colfax G., Swanson M., Chesney M. A.—Adherence and effectiveness of protease inhibitors in clinical practice in 5^{th} Conf. Retrovir. Oppor. Infect., San Francisco, 1998 Abstracts, e Eldred L.—Adherence in the era of protease inhibitors—John Hopkins AIDS Service). This percentage is considered very low for a fatal disease, especially when considering that those reports had exclusively disclosed the adherence from patients to the amount of medicines recommended.

In particular, one of the components from the anti-aids cocktail corresponds to a special class of drugs, which acts by inhibiting the retroviral protease. Protease inhibitors are substances with high molecular weight, normally lipophilic, slightly soluble in water and usually present low absorption and low bioavailability “in vivo”. Due those characteristics, elevated and frequent dosages of these substances are necessary to maintain an ideal therapeutic circulating level of the drug in the organism.

N-tert-butyldecahydro-2-[2(R)hydroxy-4-phenyl-3-(S)-[[N-(2-quinolylcarbonyl)-L-asparaginyl]amine]butyl]-(4aS,8aS)-isoquinolone-3(S)-carboxamide, also known by its generic name saquinavir, was originally developed for the treatment of AIDS as a pharmaceutical composition using its mesylate salt. Some years after its launch, it was observed that patients treated with this medicine had presented higher incidence of virus resistance development when compared to patients treated by other protease inhibitors. This feature was attributed to the poor and irregular absorption of this drug by the organism when using the pharmaceutical composition originally developed. Among several critical features considered about the composition, the presence of saquinavir in a solid form was considered to be the determinant cause for its low therapeutic efficacy.

The answer found for improving the biological activity of saquinavir was the development of a new composition, where it could be administered in a soluble way. Today saquinavir is marketed as its new composition known by the name of FORTOVASE™.

In accordance to FORTOVASE™'s monograph from the European Medicines Agency (EMEA) it is necessary to use saquinavir as an amorphous powder for the preparation of this composition, because of the low solubility of saquinavir in the excipients employed. This requires the use of a controlled crystallization technique in order to avoid the emergence of well-defined crystals when considering its industrial preparation. Besides this important technical disadvantage, that demands the establishment of selective crystallization parameters to obtain this amorphous form, another much more critical problem resides on the physical behavior of this composition. This composition that is marketed within soft gelatin capsules, most of the times does not remain as an ideal liquid, changing itself into a thick gel. The emergence of this thick gel is aleatory and unpredictable and capsules affected by this problem present significant differences of “in vitro” dissolution when compared with normal capsules.

The physical instability of the current saquinavir composition interferes in the rate of availability or drug liberation rate into the organism, as the capsules with this gelling problem will dissolve in different rates from the normal capsules, implicating in unpredictable differences of saquinavir bioavailability to someone in treatment. This problem is unacceptable considering the treatment of a viral infection where the erratic suppression of the virus can lead to the development of virus-resistance.

In the literature it is possible to find several references of pharmaceutical compositions for administering protease inhibitors. Among them there is the application that references FORTOVASE™, WO 96/39142, which describes the administration of some protease inhibitors in a composition comprising a triglyceride, which is a mixture of mono and diglycerides of fatty acids (i.e. capric and caprilic). Studies about the performance of this composition were done using as a protease inhibitor saquinavir as its base form only, and the composition was evaluated by comparing it with the saquinavir mesylate composition vehicled in hard gelatin capsules. As a result, soft gelatin capsules with saquinavir as its base form formulated in a concentrate microemulsion in the selected triglycerides showed a better bioavailability when compared to the hard gelatin capsule formulation with saquinavir as its mesylate salt, besides the better solubility of this salt form. The commercial realization of this invention is a soft gelatin capsule filled with about one gram of the concentrate microemulsion surrounded by a pellicle of soft gelatin. Due the great amount of the excipients necessary to dissolve the quantity of saquinavir per capsule (200 mg of saquinavir base), the capsule is very huge difficulting the swallowing of it by patients, in special children and debilitated patients. However, this inconvenience cannot be compared to the very serious instability problem of this composition described before, which can lead to an unpredictable absorption and bioavailability of saquinavir that can contribute extensively in the development of virus-resistance.

Another reference is application U.S. Ser. No. 08/754,390, which describes a pharmaceutical composition comprising ritonavir, a long chain fatty acid and an acceptable surfactant. Despites mentioning the possible use of saquinavir, this reference does not have any example about the preparation of a composition comprising saquinavir, only examples describing the composition using ritonavir as the active pharmaceutical ingredient. Experimentally, the preparation of a saquinavir concentrate using that predicted excipients is not feasible in such high concentrations because it is impossible to achieve its complete dissolution and there is a considerably decomposition of their constituents derived from a long period of intense heating that the composition has to be submited.

U.S. Pat. No. 6,299,906 describes a process for the preparation of submicronized particles of some biologically active compounds that are normally slightly soluble, among them saquinavir, in order to increase considerably their solubility. The process consists in the dissolution of the biologically active substance under elevate pressures within a compressed gas, liquid or supercritical fluid containing a surface modifier, and expand the compressed solution as fast as possible in order to crystallize the dissolved compound. This procedure demands the acquisition of very expensive equipments and do not enhance considerably the solubility of saquinavir. This patent does not describe any pharmaceutical composition using this submicronized saquinavir either.

Application WO 98/57648 describes methods to enhance the bioavailability of crystalline polymorphs from several compounds, among them saquinavir. The technique comprises the manufacture of nanoparticles with an average size inferior than 400 nm. The authors claims that because of the reduced size of the particles, pharmaceutical compositions manufacture with the biologically active agent becomes easier and the composition more bioavailable, mainly when presented as suspensions. As in the case of the above reference, this kind of process requires the inclusion of an extra step in the manufacture, besides the need of special equipments. There is not any suggestion for prepararing a pharmaceutical composition, as well as any test or result that demonstrates its effectiveness on enhancing saquinavir bioavailability.

There are also several references describing the preparation of pre-concentrate microemulsions suitable for the encapsulation in soft gelatin capsules, but none of those references describes compositions containing saquinavir.

One of the objectives of this invention is a pharmaceutical composition comprising a stable pre-concentrate microemulsion wherein saquinavir is soluble, suitable for the encapsulation in soft gelatin capsules or hard gelatin capsules for oral administration in the treatment of AIDS.

It is also another objective of the present invention to demonstrate that the pharmaceutical composition of the present invention presents a bioavailability profile much higher than the available marketed composition, beeing possible to use an adequate therapeutic dose considerably inferior than the therapeutic dose necessary until now.

Another objective of the present invention is the process for preparing a pharmaceutical composition comprising a concentrate microemulsion of saquinavir, a process that allows to obtain a concentrate microemulsion with high concentration of the active pharmaceutical ingredient, allowing the preparation of capsules or microcapsules comprising the adequate therapeutically amount of saquinavir.

Nowadays saquinavir in its marketed pharmaceutical composition as soft gelatin capsules must be administrated in a daily dosage of 3,600 mg, distributed in three intakes of 1,200 mg each. Soft gelatin capsules available in the market comprise saquinavir in an amount of 200 mg/capsule, being higher concentrations not prepared because it is impossible to dissolve or to prepare a stable pharmaceutical emulsion with the excipients proposed. So, the therapy consists in the ingestion of six capsules three times a day, making up a total amount of eighteen capsules taken a day. By this regularity, a treated patient must have taken an amount of 540 capsules at the end of a month, an extremely elevated quantity mainly when considering that besides this medicine other components of the anti-AIDS cocktail are also need to be taken.

Besides using saquinavir necessarily in its amorphous form, this composition presents other several ingredients that are necessary not only for their stabilization, but also to promote its dissolution within the composition. Due the low solubility of saquinavir within the selected excipients, the required amount necessary for its dissolution is extremely high in the adequate therapeutic dosage. By consequence, each saquinavir capsule commercially available contains the concentrate with the active ingredient in a final quantity of about 1,1000 mg of the composition comprising an amount of 200 mg of saquinavir. This amount of the composition is enclosed within oblong gelatin capsules N° 20, that present a voluminous size, making their ingestion difficult by patients submitted to therapy, mainly when considering patients with several infections of the digestive tract (normally present in acute cases of the disease) and to the children that are not able swallow them because of its huge size.

The pharmaceutical composition of the present invention comprises the following ingredients:
(i) N-tert-butyldecahydro-2-[2(R)hydroxy-4-phenyl-3-(S)-[[N-(2-quinolylcarbonyl)-L-asparaginyl]amine]butyl]-(4aS,8aS)-isoquinolone-3(S)-carboxamide (saquinavir) as its free base, or its pharmaceutical acceptable salts, as the active ingredient;
(ii) A fatty acid with a chain of C₁₂₋₁₈;
(iii) At least an alcohol of C₂₋₄;
(iv) At least a non-ionic surfactant;
(v) At least a pharmaceutical acceptable antioxidant.

The pharmaceutical composition of the present invention consists in a concentrate microemulsion containing as the active ingredient N-tert-butyldecahydro-2-[2(R)hydroxy-4-phenyl-3-(S)-[[N-(2-quinolylcarbonyl)-L-asparaginyl]amine]butyl]-(4aS,8aS)-isoquinolone-3(S)-carboxamide, which is known by the generic name of saquinavir, in its base form or as an acceptable pharmaceutical salt. Saquinavir, or a pharmaceutical acceptable salt of it, is used in a concentration preferably ranging from 10% to 80% in weight of the final composition. More preferably yet, saquinavir is used in a concentration ranging from 15% to 70% in weight of the final composition.

In this concentrate microemulsion composition the long chain fatty acid of C₁₂₋₁₈ presents the property to provide a hydrophobic medium adequate to avoid a possible precipitation of the active ingredient from the composition. The preferred fatty acid with a chain of C₁₂₋₁₈ is the oleic acid, which can be used in a concentration ranging from 20% to 80% in weight of the final composition, more preferably in a concentration ranging from 20% to 70% in weight of the final composition.

Saquinavir solubility and/or of its pharmaceutical acceptable salts is strongly favored by the additional presence of an alcohol of C₂₋₄, especially ethanol and/or propylene glycol. The combination of the fatty acid with one of this alcohols allows a medium wherein the completely dissolved active ingredient presents a great stability, specially if the composition does not have the presence of microcrystalline particles resulting from a poor dissolution of saquinavir or one of its pharmaceutical acceptable salts. The presence of microcrystalline forms in the super-concentrate mixtures like in the case of the presence invention, triggers the crystallization process of the active ingredient and this crystallization lower considerably the absorption of the active ingredient by the organism implicating directly in the bioavailability of the effective therapeutic dosage in the treatment of a viral infection. In the present invention the alcohol, preferably ethanol or propylene glycol or mixtures between them, will be used preferentially in a concentration ranging from 2% to 20% in weight of the final composition.

The composition of the present invention has its formula improved by the presence of a non-ionic surfactant selected among the polyethoxylated ethers derivatives from castor oil, preferably the polyethoxylated castor oil 35 (Chremophor EL), polyethoxylated hydrogenated castor oil 40 (Cremophor RH 40), this last one presenting the advantage of beeing practically insipid when used in oral formulations. Other adequate surfactants that may be used in the present invention are the polyoxyethylene sorbitan esters, compounds known as polysorbates. Among polysorbates used in the present invention, there are the liquid polysorbates like polysorbate 20, 40, 60 and 80. In the present invention surfactants are used in a concentration ranging from 0.1% to 30% in weight of the final composition.

In order to increase the stability of the composition of the present invention, substances known as antioxidants can be added to avoid its decomposition or accelerate degradation. Among adequate antioxidants useful to be used in the present invention are the alpha-tocopherol and the butylated hydroxytoluene (BHT). The use of alpha-tocopherol is advantageous in formulas of fatty acids because it exerts an adequate antioxidant activity to avoid the oxidation. In the composition of the present invention the antioxidant is used in a concentration ranging from 0.001% to 2.0% in weight of the final composition.

The pharmaceutical composition described in the present invention comprises a stable concentrate microemulsion wherein the active ingredient is completely dissolved. This concentrate microemulsion consists of a clear, transparent solution in the form of viscous oil.

The main characteristic of the composition of the present invention is its bioavailability profile surprisingly increased when compared to the existing marketed composition.

As disclosed before, saquinavir is a drug that its “in vivo” activity is very low due the poor bioavailability of the pharmaceutical compositions developed until now. Compared to other protease inhibitors, saquinavir is the substance that presents the lower bioavailability profile, beeing only just a small amount of the drug absorbed in the gastrointestinal tract and distributed to the patient tissues. Due its behavior, today it is necessary the ingestion of several capsules from the medicine available in the market to reach the appropriate circulating therapeutic dose.

The composition of the present invention has a bioavailability profile several times higher than all pharmaceutical formulations developed until the present moment, allowing a marked reduction in the amount of capsules necessary to be ingested everyday and/or the reduction of the capsule volume in order to facilitate its ingestion.

Considering the issue adhesion or adherence to the amount necessary of this medicine, the pharmaceutical composition of the present invention presents a bioavailability profile that contributes positively in the acceptance of the therapy by the patient. As disclosed before, a patient in treatment with saquinavir need to take up to 18 capsules of this medicine each day. The composition of the present invention allows the use of only three to five capsules of this medicine each day to reach the plasmatic levels suitable for the therapy, incisively contributing with the adherence of the patient to the amount of the drug prescribed and eliminating de discomfort of taking an excessive amount of medicines.

As innovator features of the this composition, special attention must be given to the bioavailability of saquinavir, which is at least five times higher than the composition now available in the market. Other innovative characteristic corresponds to the possibility of this composition being formulated comprising elevate quantities of saquinavir, without occurring its later precipitation or crystallization. Because of this aspect, the composition of the present invention can be formulated using saquinavir in an amount ranging from 10% to 80% in weight of the final composition. Preferably the concentration range in the present invention will be from 15% to 70% in weight of the final composition, which corresponds to a concentration ranging from 150 mg to 700 mg of saquinavir per gram of the final composition. It corresponds to a dose 350% higher than the marketed composition, when considering its higher concentration.

By all these characteristics, the administration regime of this medicine can be improved and simplified. The quantity of capsules to be ingested can be lowered and/or the capsules can be miniaturized in order to have a more appropriate size to the ingestion.

Considering that the main factor for the non-adherence or partial adherence from patients to the treatment with protease inhibitors, specially saquinavir, is the quantity of capsules to be ingested daily and their uncomfortable size for the ingestion, the pharmaceutical composition of the present invention consists in an alternative extremely favorable for the adherence of the patient to treatment. Through the improvement achieved, it is possible the administration of higher quantities of saquinavir per capsule reducing considerably the amount of capsules to be taken each time.

Other alternative to increase the adhesion of patient to treatment is the possibility to promote the manufacture of a capsule with considerably reduced size, allowing its easy administration to patients with problems in ingesting the voluminous capsules now available in the market, even allowing the treatment of children that are not treated because they are not able to ingest the already available medicine.

Another objective of the present invention is the process for preparing pharmaceutical compositions with increased bioavailability, consisting in a concentrate microemulsion of saquinavir or its pharmaceutical acceptable salts.

Due the low solubility of saquinavir and/or its pharmaceutical acceptable salts, the preparing of the pharmaceutical composition can not be executed using a direct dissolution technique, or it means, it is not possible to obtain this pharmaceutical composition in the indicated concentrations by a procedure consisting basically in the dissolution of saquinavir or one of its salts in any of its ingredients or their indicated combinations, even when the active ingredient is micronized or submitted to elevate temperatures for prolonged periods of time in order to proportionate its dissolution.

The process for preparing pharmaceutical compositions constituted by a concentrate microemulsion of N-tert-butyldecahydro-2-[2(R)hydroxy-4-phenyl-3-(S)-[[N-(2-quinolylcarbonyl)-L-asparaginyl]amine]butyl]-(4aS,8aS)-isoquinolone-3(S)-carboxamide (saquinavir), or its pharmaceutical acceptable salts, comprises the following steps:
a) Dissolving completely of N-tert-butyldecahydro-2-[2(R)hydroxy-4-phenyl-3-(S)-[[N-(2-quinolylcarbonyl)-L-asparaginyl]amine]butyl]-(4aS,8aS)-isoquinolone-3(S)-carboxamide, or its pharmaceutical acceptable salt, in a sufficient amount of the alcohol of C₂₋₄ under controlled temperature;
b) Eliminating particles by filtration;
c) Adding the fatty acid, the antioxidant and the surfactant in an appropriate amount used in the composition;
d) Evaporating the alcohol at a maximum temperature of 50° C. under reduced pressure;
e) Optionally, adding the surfactant from step (c) after the evaporation of the alcohol from step (d);
f) Adding the alcohol C₂₋₄ under stirring and in an enough amount to complete the adequate weight of the final composition.

Initially saquinavir, or its pharmaceutical acceptable salt, is completely dissolved in sufficient amount of alcohol necessary to obtain a completely clear solution, In order to avoid any degradation of the active ingredient, this dissolution is performed in a temperature ranging from 20° C. to 50° C., under stirring. To guarantee the absence of solid particles that can trigger the later precipitation process, this alcoholic solution is filtered using usual filtration techniques and to the clear resulting solution are added the fatty acid and the antioxidant, being the resulting solution concentrated at reduced pressure employing a maximum temperature of about 50° C. To the resulting concentrate is added the surfactant and the mixture is stirred until it turns into a clear oily liquid. Optionally the surfactant may be added with the fatty acid and the antioxidant together. The final correction of the composition is done by adding the alcohol until the composition reaches the weight concentration desired of saquinavir or its pharmaceutical acceptable salt.

The alcohol used in the initial dissolution of saquinavir is preferably the ethanol, because its evaporation can be easily done under low temperatures at reduced pressures employing usual industrial techniques. After the addition of the other components, the fatty acid, the antioxidant and the surfactant, the alcohol used to complete the final weight of the composition is preferably ethanol, or propylene glycol or mixtures between them.

In accordance to the process, the resulting alcoholic solution from saquinavir dissolution presents a saquinavir concentration ranging from 0.01% to 90% in weight of the final solution. The alcohol preferably employed is the ethanol, but other alcohols of C₂₋₄ may be used, namely methanol, isopropanol, propanol and butanoles. The alcohol used for preparing the saquinavir solution is preferably used in a concentration ranging from 10% to 99.99% in weight of the final solution. The temperature used for dissolving saquinavir in the alcohol ranges from 20° C. to 50° C., temperatures mild enough to avoid saquinavir degradation. More elevate temperatures may be used, taking the appropriate care to monitor saquinavir stability in the solution.

After dissolving saquinavir in the alcohol, the resulting solution is filtered using usual industrial filtration techniques. Filtration through microporous membranes is particularly interesting, eliminating eventual microparticles that could remain in the solution.

Excipients comprising the final composition are add after the filtration, being them the fatty acid of C₁₂₋₁₈, the antioxidant and the surfactant that can be add optionally in this step or after the next step consisting in the alcohol evaporation.

Alcohol elimination by evaporation is preferably performed under reduced pressure, so controlling the medium temperature avoiding degradation of the active ingredient and/or the excipients added. Alcohol elimination is performed until the composition reaches its appropriate weight or it can be removed until the composition reaches a weight lower than desired, the final composition weight being reached by adding one or more alcohols of C₂₌₄.

The product resulting from the process disclosed above presents as its final composition saquinavir in a concentration ranging from 10% to 80%, preferably in a concentration ranging from 15% to 75% in weight of the final composition. The fatty acid of chain C₁₂₋₁₈ is present in a concentration ranging from 20% to 80% in weight of the final composition, preferably in a concentration ranging from 20% to 70% in weight of the final composition. It presents one or more alcohols of C₂₋₄ in a concentration ranging from 2.0% to 20% in weight of the final composition. It presents the non-ionic surfactant selected among polyethoxylated castor oil derivatives, preferably polyethoxylated castor oil 35 (Cremophor 35) or polyethoxylated hydrogenated castor oil 40 (Cremophor RH 40) or among polyoxyethylene sorbitan esters (polysorbates), preferably liquid polysorbates at ambient temperatures like polysorbates 20, 40, 60 or 80 in a concentration ranging from 0.1% to 30% in weight of the final composition. Finalizing, the product resulting from the described process presents as antioxidant the alpha-tocopherol or butylated hydroxytoluene in a concentration ranging from 0.001% to 2.0% in weight of the final composition.

By this procedure it is possible to obtain a pharmaceutical composition of a stable concentrate microemulsion of saquinavir, or one of its acceptable salt, wherein the active ingredient is completely dissolved, without the presence of saquinavir microcrystalline forms, that are able to trigger the crystallization of the active ingredient on standing, which would interfere in the absorption and the bioavailability of the medicine.

By using this process there is no need to use saquinavir or one of its pharmaceutical acceptable salts in a special crystalline form as, for example, in an amorphous or micronized form in order to allow its complete dissolution in the selected excipients. Any crystalline form can be used without interfering on its stability over crystallization from the final composition.

Additionally, by this technical advance, it is possible to prepare highly concentrate compositions, impossible to prepare by direct dissolution procedures using the excipients described.

The concentrate microemulsion resulting from this process presents high stability over precipitation and/or crystallization and over changes in its physical state. Even when the composition is submitted to low temperatures (4° C. to 8° C.) for long periods of time, there is no sign of gel formation or the appearance of crystalline or microscristalline formation, which would trigger the precipitation or crystallization of the active ingredient, and such event would damage the absorption and bioavailability of the protease inhibitor, which would not be soluble in the composition.

The stability of the present invention composition over crystallization of saquinavir grants its delivery or liberation in the absorption place in a soluble form, adequate to its prompt absorption by the organism.

Stability monitoring of the concentrate microemulsion composition shows a reliable result on considering the ability of keeping the active ingredient in a soluble state suitable for the prompt absorption of the drug by the organism. During the studies it was not observed the formation or transformation of the solution into a jelly composition, as it happens with the marketed capsules available today, demonstrating that the pharmaceutical composition of the present invention presents a physical form stable and adequate to the liberation and dissolution in a constant rate.

The composition of the present invention comprising a concentrate microemulsion can be employed in a final pharmaceutical presentation consisting by soft or hard gelatin capsules.

In a preferential realization of the present invention, a pharmaceutical composition consisting of a concentrate microemulsion disclosed abovr is encapsulated in soft gelatin capsules that present uniform liberation properties of its contents inside the gastrointestinal tract, as well presenting a better receptivity from the patient because of its elastic properties that allows an easier ingestion.

The technique for preparing the soft gelatin capsule is very well known basically consisting on using gelatin, plasticizing agent and water in definite proportions. Additionally the capsule material may contain additives like inks, pigments and flavors, among others. The manufacture of soft gelatin capsules comprises several techniques, like, for example, a process with or without sewing, rotatory, using specific machinery, among others. Soft gelatin capsules used in this invention as a film for covering the concentrate pharmaceutical formulation consist in pharmaceutical gelatin, glycerol, propylparabene, titanium dioxide and water, and they were prepared by conventional technique.

As a general rule the composition of the present invention can be submitted to all of the existing processes of producing soft gelatin capsules since they do not considerably interfere with the composition, it means, that such process of producing does not considerably change the ratio among its ingredients by evaporation because heat exposition, drying processes or any other kind of processing.

The following examples are illustrative, but not exhaustive about the possibilities of the composition of the present invention and its process for preparing, as well the tests that demonstrate its stability and the maintaining of its properties of a soluble concentrate microemulsion of saquinavir on standing, and its improved bioavailability profile.

### EXAMPLE 1a

### Preparation of a 20% Saquinavir Composition

In a 2 L reactor add 200 g (20.00%) of saquinavir and 1,000 mL of absolute ethanol. Keep the system under stirring at a temperature up to 50° C. until all solids dissolution. This solution is filtered to eliminate solid particles and to the filtrate are add oleic acid (526.2 g-52.2%) and tocopherol (7.44 g-0.744%). This mixture is stirred for 5 minutes and then is vacuum concentrated at a temperature up to 50° C. To the resulting concentrate, polyethoxylated castor oil 35 is added (141.8-14.18%) and the final weight of the solution is brought to 1,000 g with absolute ethanol, if necessary.

By this way prepared, the final composition presents a concentration of 200 mg of saquinavir for each 1 g of the composition. It is a clear and yellow oily liquid. When stored under refrigeration (4° C. to 8° C.) for 120 days it does not present crystals or any dispersed solid formations, maintaining its clear aspect.

### EXAMPLE 1b

### Preparation of a 20% Saquinavir Composition with Propylene Glycol

For preparing a concentrate composition of saquinavir comprising propylene glycol as the alcoholic excipient, it is used the procedure described in Example 1a, but replacing the alcohol used to complete the final weight of the resulting composition by propylene glycol.

### EXAMPLE 2a

### Preparation of a 40% Saquinavir Composition

In a 5 L reactor add 400 g (40%) of saquinavir and 2,000 mL of absolute ethanol. Keep the system under stirring in a temperature up to 50° C. until all solids dissolution. This solution is filtered to eliminate solid particles and to the filtrate are added the oleic acid (370 g-37%), the tocopherol (7.44 g-0.744%) and the polyethoxylated castor oil 35 (100 g-10%). This mixture is stirred for 5 minutes and then is concentrated under vacuum at a temperature up to 50° C. The final weight of the solution is corrected to 1,000 g with absolute ethanol if necessary.

By this way prepared, the final composition presents a concentration of 400 mg of saquinavir for each 1 g of the composition. It is a clear and yellow oily liquid. When stored under refrigeration (4° C. to 8° C.) for 120 days it does not present crystals or any dispersed solid formations, maintaining its clear aspect.

### EXAMPLE 2b

### Preparation of a 40% Saquinavir Composition with Propylene Glycol

For preparing a concentrate composition of saquinavir comprising propylene glycol as the alcohol as excipient, it is used the procedure described in Example 2a, but replacing the alcohol used to complete the final weight of the resulting composition by propylene glycol.

### EXAMPLE 3a

### Preparation of a 60% Saquinavir Composition

In a 5 L reactor add 600 g (60%) of saquinavir and 3,000 mL of absolute ethanol. Keep the system under stirring in a temperature ranging from 20° C. to 50° C. untill all solids dissolution. This solution is filtered to eliminate solid particles and to the filtrate are added the oleic acid (200 g-20%) and the tocopherol (7.44 g-0.744%). This mixture is stirred for 5 minutes and then is concentrated under vacuum at a temperature up to 50° C. To this concentrate is added the polyethoxylated castor oil 35 (150 g-15%) and the final weight of the solution is corrected to 1,000 g with absolute ethanol, if necessary.

By this way prepared, the final composition presents a concentration of 600 mg of saquinavir for each 1 g of the composition. It is a clear and yellow oily liquid. When stored under refrigeration (4° C. to 8° C.) for 120 days it does not present crystals or any dispersed solid formations, maintaining its clear aspect.

### EXAMPLE 3b

### Preparation of a 60% Saquinavir Composition with Propylene Glycol

For preparing a concentrate composition of saquinavir comprising propylene glycol as the alcohol as excipient, it is used the procedure described in Example 3a, but replacing the alcohol used to complete the final weight of the resulting composition by propylene glycol.

By using the process described in the above examples there were prepares the compositions disclosed in the Table 1 below.

TABLE 1
Examples of saquinavir concentrate compositions
prepared accordingly with the present invention

| Composition | | Used quantities | |
|---|---|---|---|
| C1 | Saquinavir base | 200 | g |
| | Oleic acid | 526.2 | g |
| | Tocopherol | 7.44 | g |
| | Polyethoxylated castor oil 35 | 141.8 | g |
| | Absolute Ethanol | s.q.t. 1,000 | g |
| C2 | Saquinavir base | 200 | g |
| | Oleic acid | 450 | g |
| | Tocopherol | 5.0 | g |
| | Polyethoxylated castor oil 35 | 225 | g |
| | Absolute Ethanol | s.q.t. 1,000 | g |
| C3 | Saquinavir mesylate | 228.65 | g |
| | Oleic acid | 505 | g |
| | Tocopherol | 7.44 | g |
| | Polyethoxylated castor oil 35 | 134 | g |
| | Absolute Ethanol | s.q.t. 1,000 | g |
| C4 | Saquinavir base | 600 | g |
| | Oleic acid | 555 | g |
| | Tocopherol | 7.44 | g |
| | Polyethoxylated castor oil 35 | 150 | g |
| | Absolute Ethanol | s.q.t. * 1,500 | g |
| C5 | Saquinavir base | 600 | g |
| | Oleic acid | 200 | g |
| | Tocopherol | 7.44 | g |
| | Polyethoxylated castor oil 35 | 150 | g |
| | Absolute Ethanol | s.q.t. * 1,000 | g |
| C6 | Saquinavir Mesylate | 685.96 | g |
| | Oleic acid | 500 | g |
| | Tocopherol | 7.44 | g |
| | Polyethoxylated castor oil 35 | 120 | g |
| | Absolute Ethanol | s.q.t. 1,500 | g |
| C7 | Saquinavir base | 600 | g |
| | Oleic acid | 230 | g |
| | Tocopherol | 3.7 | g |
| | Polyethoxylated castor oil 35 | 125 | g |
| | Absolute Ethanol | s.q.t. 1,000 | g |
| C8 | Saquinavir base | 600 | g |
| | Oleic acid | 555 | g |
| | Tocopherol | 7.44 | g |
| | Polyethoxylated castor oil 35 | 150 | g |
| | Propyleneglycol | s.q.t. 1,500 | g |
| C9 | Saquinavir base | 600 | g |
| | Oleic acid | 555 | g |
| | Tocopherol | 7.44 | g |
| | Polyethoxylated castor oil 35 | 200 | g |
| | Absolute Ethanol | s.q.t. 1,500 | g |
| C10 | Saquinavir Masylate | 685.96 | g |
| | Oleic acid | 430 | g |
| | Tocopherol | 7.44 | g |
| | Polyethoxylated castor oil 35 | 200 | g |
| | Absolute Ethanol | s.q.t. 1,500 | g |
| C11 | Saquinavir base | 600 | g |
| | Oleic acid | 200 | g |
| | Tocopherol | 7.44 | g |
| | Polyethoxylated castor oil 35 | 68 | g |
| | Absolute Ethanol | s.q.t. 1,000 | g |
| C12 | Saquinavir base | 600 | g |
| | Oleic acid | 555 | g |
| | Tocopherol | 7.44 | g |
| | Polyethoxylated castor oil 35 | 150 | g |
| | Propylenoglycol/ethanol (1:1 volume) | s.q.t. * 1,500 | g |
| C13 | Saquinavir base | 600 | g |
| | Oleic acid | 200 | g |
| | Tocopherol | 7.44 | g |
| | Polyethoxylated castor oil 35 | 150 | g |
| | Propyleneglycol/ethanol (1:1 volume) | s.q.t. * 1,000 | g |
| C14 | Saquinavir base | 600 | g |
| | Oleic acid | 750 | g |
| | Tocopherol | 7.44 | g |
| | Polyethoxylated castor oil 40 | 20 | g |
| | Absolute ethanol | s.q.t. * 1,500 | g |
| C15 | Saquinavir base | 600 | g |
| | Oleic acid | 555 | g |
| | Tocopherol | 7.44 | g |
| | Polyssorbate 40 | 150 | g |
| | Absolute ethanol | s.q.t. * 1,500 | g |

s.q.t.—Suficient Quantity To complete the desired weight.

Bioavailability Comparative Study

The bioavailability profile of composition C1 from the present invention was evaluated in human volunteers in comparison with FORTOVASE™ composition.

The study consisted in the administration of a single dose of 200 mg of saquinavir from the composition C1 of the present invention and from the FORTOVASE™ composition.

Individuals were submitted to a two confinement periods of 25 hours, with a rest period of one week between them, and all individuals received both compositions in a random way according to the design of the study.

Twelve healthy volunteers from both sexes were used in the study, each one of them receiving a 200 mg FORTOVASE™ composition or a 200 mg of composition C1. Volunteers received the medication in a fasting state followed by 200 mL of water. Fasting was established by a two hours period after the administration of the dosae, and after that it was served a standard breakfast free from xanthine. Five hours after the administration of the drugs another meal was offered, 8 hours after administration was offered a lunch and another meal at 11 hours after administration, all the meals free from xanthine. Liquid refreshments were free allowed, but xanthine-containing liquids were prohibited.

Alcohol use was forbidden from the last 48 hours before the beginning of the study. Use of cigarettes, cigars and similar were not allowed during the study.

Saquinavir plasma concentration was determined by high performance liquid chromatography with mass spectrometry (LC-MS-MS). Blood samples for the assay of the drug in the plasma were collected at 0.33; 0.67; 1; 1.33; 1.67; 2; 2.33; 2.67; 3; 3.5; 4; 4.5; 5; 5.5; 6; 7; 8; 10 and 12 hours after the administration of the medicine.

The maximum concentration (Cₘₐₓ) observed in the plasma and the time to achieve the maximum concentration were determined for each composition. Areas under the time-concentration curve were calculated by using the linear regression log trapezoidal, except for AUC₀₋₁₂ₕ, were it was used the trapezoidal method.

Table 2 below summarizes the pharmacokinetics parameters from the inventive composition C1 against the reference medicine FORTOVASE™.

TABLE 2
Results from the comparative bioavailability study.

| | FORTOVASE ® | Inventive composition |
|---|---|---|
| Cₘₐₓ | 11.26 | 64.48 |
| Tₘₐₓ | 0.67 | 0.67 |
| AUC₀₋₁₂ | 13.20 | 69.58 |

Data from Table 2 reveal that the inventive composition C1 presents a Cₘₐₓ almost six times higher and an AUC₀₋₁₂ₕ five times higher than the same parameters found for the reference composition (FORTOVASE™). These data are surprisingly, indicating an increased bioavailability when comparing the inventive composition against the reference composition.

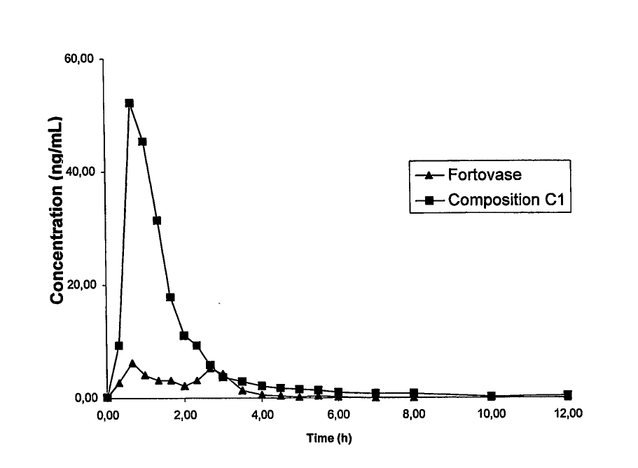

FIG. 1 presents the comparative plot of the mean concentration of saquinavir from the present invention composition C1, and the saquinavir from reference FORTOVASE™ composition over time.

1-36. (canceled)

37. A pharmaceutical composition for oral administration of saquinavir comprising:
(i) saquinavir or its pharmaceutical acceptable salts as the active ingredient in a concentration ranging from 10% to 80% in weight of the final composition;
(ii) a long chain fatty acid of C₁₂₋₁₈ in a concentration ranging from 20% to 80% in weight of the final composition;
(iii) at least an alcohol of chain C₂₋₄ in a concentration ranging from 2% to 20% in weight of the final composition;
(iv) a non-ionic surfactant in a concentration ranging from 0.1% to 30% in weight of the final composition;
(v) a pharmaceutical acceptable antioxidant in a concentration ranging from 0.001% to 2.0% in weight of the final composition.

38. The pharmaceutical composition of claim 37, wherein saquinavir, or its pharmaceutical acceptable salts, is employed in a concentration ranging from 15% to 70% in weight of the final composition.

39. The pharmaceutical composition of claim 37, wherein the fatty acid of C₁₂₋₁₈ is oleic acid, used in a concentration ranging from 20% to 70% in weight of the final composition.

40. The pharmaceutical composition of claim 37, wherein the alcohol of C₂₋₄ is ethanol or propyleneglicol or mixtures thereof, in a concentration ranging from 2% to 20% in weight of the final composition.

41. The pharmaceutical composition of claim 37, wherein the non-ionic surfactant is selected among polyethoxylated derivatives from castor oil and polyoxyethylene sorbitan esters (polysorbates) in a concentration ranging from 0.1% to 30% in weight of the final composition.

42. The pharmaceutical composition of claim 41, wherein the polyethoxylated derivatives from castor oil are polyethoxylated castor oil 35 (Cremophor EL) or polyethoxylated hydrogenated castor oil 40 (Cremophor RH 40), in a concentration ranging from 0.1% to 30% in weight of the final composition.

43. The pharmaceutical composition of claim 41, wherein the polyoxyethylene sorbitan esters are the liquid polysorbates like as polysorbate 20, 40, 60 and 80, in a concentration ranging from 0.1% to 30% in weight of the final composition.

44. The pharmaceutical composition of claim 37, wherein the pharmaceutical acceptable antioxidant is alpha-tocopherol or butylated hidroxytoluene, in a concentration ranging from 0.001% to 2.0% in weight of the final composition.

45. The pharmaceutical composition according to claim 37, characterized by consisting of a stable concentrate microemulsion wherein the active ingredient saquinavir, or its pharmaceutical acceptable salts, is soluble.

46. The pharmaceutical composition of claim 45, which is fractionated in single doses in the form of soft gelatin capsules for oral administration in the treatment of AIDS.

47. The pharmaceutical composition according to claim 45, which is fractionated in single doses in the form of hard gelatin capsules for oral administration in the treatment of AIDS.

48. The pharmaceutical composition according to claim 37 in which the bioavailability of saquinavir, when measured by AUC and Cₘₐₓ parameters, is at least 5 times higher than the same dosage from the reference composition.

49. A process for preparing a pharmaceutical composition for oral administration of saquinavir in accordance with the claim 37 which comprises the following steps:
(a) Completely dissolving saquinavir, or its pharmaceutical acceptable salt, in a sufficient amount of the alcohol of C₂₋₄ under controlled temperature;
(b) Eliminating particles by filtration;
(c) Adding the fatty acid of chain C₁₂₋₁₈, the antioxidant and the non-ionic surfactant in an appropriate amount used in the composition;
(d) Evaporating the alcohol at a maximum temperature of 50° C. under reduced pressure;
(e) Optionally, adding the non-ionic surfactant from step (c) after the evaporation of the alcohol from step (d);
(f) Adding the alcohol C₂₋₄ under stirring and in an enough amount to complete the adequate weight of the final composition;
(g) Resulting in the final composition in the form of a stable concentrate microemulsion wherein the saquinavir form is soluble.

50. The process of claim 49, wherein in step (a) saquinavir, or its pharmaceutical acceptable salts, is in the crystalline, amorphous, micronized or mixtures of that forms, in a concentration ranging from 0.01% to 90% in weight of the final solution.

51. The process of claim 49, wherein the alcohol of chain C₂₋₄ in step (a) is used in a concentration ranging from 10% to 99.99% in weight of the final solution.

52. The process of claim 51, wherein the alcohol of chain C₂₋₄ is ethanol.

53. The process according with claim 49, wherein the temperature in step (a) ranges from 20° C. to 50° C.

54. The process of claim 49, wherein the fatty acid of chain C₁₂₋₁8 used in step (c) is oleic acid.

55. The process of claim 49, wherein the antioxidant used in step (c) is tocopherol, butylated hydroxytoluene or mixtures thereof.

56. The process of claim 49, wherein the non-ionic surfactant used in step (c) or (e) is selected among polyethoxylated derivatives from castor oil and polyoxyethylene sorbitan esters (polysorbates).

57. The process of claim 56, wherein the non-ionic surfactant used in step (c) or (e) is polyethoxylated castor oil 35 (Cremophor EL) or polyethoxylated hydrogenated castor oil 40 (Cremophor RH 40).

58. The process of claim 56, wherein the non-ionic surfactant used in step (c) or (e) is a liquid polysorbate like as polysorbate 20, 40, 60 and 80.

59. The process of claim 49, wherein the alcohol of chain C₂₋₄ used in step (f) is ethanol, or propyleneglycol, or mixtures thereof.

60. The process of claim 49, wherein the resulting product in the step (g) presents saquinavir, or its pharmaceutical acceptable salts, in a concentration ranging from 10% to 80% in weight of the final pharmaceutical composition.

61. The process of claim 60, wherein the resulting product in the step (g) presents saquinavir, or its pharmaceutical acceptable salts, in a concentration ranging from 15% to 70% in weight of the final pharmaceutical composition.

62. The process of claim 60, wherein the resulting product in the step (g) contains the fatty acid of chain C₁₂₋₁₈ in a concentration ranging from 20% to 80% in weight of the final pharmaceutical composition.

63. The process of claim 49, wherein the resulting product in the step (g) contains oleic acid as the fatty acid of chain C₁₂₋₁₈, in a concentration ranging from 20% to 70% in weight of the final pharmaceutical composition.

64. The process of claim 49, wherein the resulting product in the step (g) contains ethanol, or propyleneglycol or mixtures thereof as the alcohol of chain C₂₋₄, in a concentration ranging from 2.0% to 20% in weight of the final pharmaceutical composition.

65. The process of claim 49, wherein the resulting product in the step (g) contains a non-ionic surfactant selected from the group constituted of polyethoxylated castor oil 35 (Cremophor 35), polyethoxylated hydrogenated castor oil 40 (Cremophor RH 40), polysorbate 20, polysorbate 40, polysorbate 60, polysorbate 80 or mixtures thereof, in a concentration ranging from 0.1% to 30% in weight of the final pharmaceutical composition.

66. The process of claim 49, wherein the resulting product in the step (g) contains alpha-tocopherol or butylated hydroxytoluene as the antioxidant, in a concentration ranging from 0.001% to 2.0% in weight of the final pharmaceutical composition.

67. The process of claim 49, wherein the resulting product in the step (g) is in a suitable form to be encapsulated in hard or soft gelatin capsules for the oral administration in AIDS treatment.

68. A method to increase bioavailability of saquinavir, or its pharmaceutical acceptable salts, which consists in administering to a patient during the therapy a pharmaceutical composition prepared according to claim 49.
